SEQUENCE LISTING (48 sequences)

SEQ ID NO: 1 - protein, 657 aa, artificial
  fusion protein, mature protein
AGCKNFFWKTFTSCAEAAAKEAAAKEAAAKEAAAKADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKEAAAKEAAAKEAAAKAAGCKNFFWKTFTSC

SEQ ID NO: 2 - protein, 621 aa, artificial
  fusion protein, mature protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKEAAAKEAAAKEAAAKAAGCKNFFWKTFTSC

SEQ ID NO: 3 - protein, 1203 aa, artificial
  fusion protein, mature protein
HHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - protein, 1222 aa, artificial
  fusion protein, mature protein
HHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 1213 aa, artificial
  fusion protein, mature protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 8 aa, Artificial
  linker
GGGGGGGG

SEQ ID NO: 7 - protein, 614 aa, artificial
  fusion protein, mature protein
AGCKNFFWKTFTSCGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 621 aa, artificial
  fusion protein, mature protein
AGCKNFFWKTFTSCAEAAAKEAAAKEAAAKEAAAKADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 613 aa, artificial
  fusion protein, mature protein
HHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSC

SEQ ID NO: 10 - protein, 613 aa, artificial
  fusion protein, mature protein
AGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSHHHHHH

SEQ ID NO: 11 - protein, 604 aa, artificial
  fusion protein, mature protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSC

SEQ ID NO: 12 - protein, 604 aa, artificial
  fusion protein, mature protein
AGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - protein, 623 aa, artificial
  fusion protein, mature protein
AGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 14 - protein, 661 aa, artificial
  fusion protein, mature protein
AGCKNFFWKTFTSCGGGGSAGCKNFFWKTFTSCGGGGSAGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 15 - protein, 614 aa, artificial
  fusion protein, mature protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAGCKNFFWKTFTSC

SEQ ID NO: 16 - protein, 629 aa, artificial
  fusion protein, mature protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSAGCKNFFWKTFTSC

SEQ ID NO: 17 - protein, 14 aa, Homo sapiens
AGCKNFFWKTFTSC

SEQ ID NO: 18 - protein, 28 aa, Homo sapiens
SANSNPAMAPRERKAGCKNFFWKTFTSC

SEQ ID NO: 19 - protein, 585 aa, Homo sapiens [sequence omitted]

SEQ ID NO: 20 - protein, 22 aa, artificial
  signal peptide for fusion protein
MDMRVPAQLLGLLLLWLRGARC

SEQ ID NO: 21 - protein, 20 aa, artificial
  linker
APAPAPAPAPAPAPAPAPAP

SEQ ID NO: 22 - protein, 40 aa, artificial
  linker
APAPAPAPAPAPAPAPAPAPAPAPAPAPAPAPAPAPAPAP

SEQ ID NO: 23 - DNA, 42 bp, Homo sapiens
gctggctgcaagaatttcttctggaagactttcacatcctgt

SEQ ID NO: 24 - DNA, 84 bp, Homo sapiens
tctgctaactcaaacccggctatggcaccccgagaacgcaaagctggctgcaagaatttcttctggaagactttcacatcctgt

SEQ ID NO: 25 - DNA, 1755 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 26 - DNA, 5428 bp, artificial
  mammalian expression vector
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 27 - DNA, 9276 bp, artificial
  yeast expression vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattccctagggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 28 - DNA, 45 bp, artificial
  flexible, extended linker
ggtggaggtggctcaggtggaggtggctcaggtggaggtggctca

SEQ ID NO: 29 - DNA, 66 bp, artificial
  alpha helical linker
gctgaagctgcagccaaagaagctgcagccaaagaggccgcagctaaggaagccgcagcaaaagct

SEQ ID NO: 30 - protein, 22 aa, artificial
  alpha helical peptide linker
AEAAAKEAAAKEAAAKEAAAKA

SEQ ID NO: 31 - protein, 5 aa, artificial
  flexible, extended peptide linker
GGGGS

SEQ ID NO: 32 - protein, 16 aa, artificial
  linker with thrombin cleavage site
GGGGSLVPRGSGGGGS

SEQ ID NO: 33 - protein, 6 aa, artificial
  linker
GSGSGS

SEQ ID NO: 34 - protein, 15 aa, artificial
  linker with thrombin cleavage site
GGGGSLVPRGSGGGG

SEQ ID NO: 35 - protein, 16 aa, artificial
  linker with thrombin cleavage site
GGGGSLVPRGSGGGGS

SEQ ID NO: 36 - protein, 11 aa, artificial
  lilnker
GGSGGHMGSGG

SEQ ID NO: 37 - protein, 11 aa, artificial
  linker
GGSGGSGGSGG

SEQ ID NO: 38 - protein, 11 aa, artificial
  linker
GGSGGHMGSGG

SEQ ID NO: 39 - protein, 5 aa, artificial
  linker
GGSGG

SEQ ID NO: 40 - protein, 16 aa, artificial
  linker
GGGGSLVPRGSGGGGS

SEQ ID NO: 41 - protein, 8 aa, artificial
  linker
GGSGGGGG

SEQ ID NO: 42 - protein, 8 aa, artificial
  linker
GSGSGSGS

SEQ ID NO: 43 - protein, 18 aa, artificial
  linker
GGGSEGGGSEGGGSEGGG

SEQ ID NO: 44 - protein, 8 aa, artificial
  linker
AAGAATAA

SEQ ID NO: 45 - protein, 5 aa, artificial
  linker
GGGGG

SEQ ID NO: 46 - protein, 5 aa, artificial
  linker
GGSSG

SEQ ID NO: 47 - protein, 11 aa, artificial
  linker
GSGGGTGGGSG

SEQ ID NO: 48 - protein, 26 aa, artificial
  linker
GSGSGSGSGGSGGSGGSGGSGGSGGS

(19) United States
(12) Patent Application Publication
(10) Pub. No.: US 2017/0002057 A1
(43) Pub. Date: Jan. 5, 2017

(54) COMPOSITIONS CONTAINING FUSION PROTEIN OF ALBUMIN AND ANALOGS THEREOF, METHODS FOR MAKING AND USING THE SAME

(71) Applicant: NAL PHARMACEUTICAL GROUP LIMITED, Hong Kong (CN)

(72) Inventors: Y. Joseph MO, Princeton, NJ (US); Chun Kwong CHU, Hong Kong (CN)

(73) Assignee: NAL PHARMACEUTICAL GROUP LIMITED, Hong Kong (CN)

(21) Appl. No.: 15/249,346

(22) Filed: Aug. 26, 2016

Related U.S. Application Data
(63) Continuation-in-part of application No. PCT/US2016/019950, filed on Feb. 26, 2016.
(60) Provisional application No. 62/121,487, filed on Feb. 26, 2015.

(30) Foreign Application Priority Data
Feb. 26, 2016 (TW) 105106088

(51) Int. Cl. C07K 14/765; C07K 7/06

(52) U.S. Cl. CPC C07K 14/765; C07K 7/06; C07K 2319/00; A61K 38/00

(57) ABSTRACT

The invention is related to fusion proteins of human somatostatin (e.g., SST-14 or SST-28) and human serum albumin, comprising a region at least 85% homologous to human somatostatin and a region at least 85% homologous to human serum albumin or a region with a partial amino acid sequence of human serum albumin, wherein linker peptide sequences may be present between somatostatin and somatostatin moieties or somatostatin and albumin moieties. Also disclosed are constructs wherein the somatostatin moiety contains multiple tandem repeats of a somatostatin sequence. In selected embodiments, the orientation of the somatostatin and albumin moieties can be varied, and such sequences may impact the binding and efficacy of the disclosed fusion proteins. Also disclosed are methods of making and using the aforementioned constructs. The somatostatin-albumin fusion protein demonstrated enhanced stability when incubated in rat plasma in vitro and prolonged plasma half-life in vivo compared with free somatostatin.

### CROSS-REFERENCE TO RELATED APPLICATIONS

This application is a continuation-in-part of PCT/US2016/019950, which claims the benefit of priority from U.S. Provisional Patent Application Ser. No. 62/121,487 filed on Feb. 26, 2015, the contents of which are incorporated herein by reference. This application also claims benefit from Taiwanese Patent Application No. 105106088 filed Feb. 26, 2016, which also claims benefit of priority from U.S. Provisional Patent Application No. 62/121,487 filed on Feb. 26, 2015, the contents of which are incorporated herein by reference.

### FIELD OF INVENTION

The present invention relates to a fusion protein comprising a somatostatin, or its analogue or derivatives, a linker or spacer and an albumin, or its analogue or variant.

The present invention also relates to recombinant fusion proteins containing a human serum albumin moiety, and a somatostatin moiety, separated by a spacer sequence and analogues thereof.

### BACKGROUND OF THE INVENTION

Somatostatin (“SST”) is a secretory product of a variety of endocrine and non-endocrine tissues and is widely distributed throughout the body. Somatostatin inhibits pituitary, pancreatic, and gastrointestinal hormone secretion release, as well as cytokine production, intestinal motility and absorption, vascular contractility, and cell proliferation. Recent studies have found that SST has use as a treatment for cancer, inhibiting tumor growth, inhibiting the proliferation of endocrine tumors, and many other solid tumors, such as breast cancer, colorectal cancer, liver cancer, lung cancer, endocrine cancer, neuroendocrine cancers, pancreatic cancer and prostate cancer. The somatostatin molecule has two biologically active forms: somatostatin-14 (SST-14), the cyclic tetradecapeptide, and somatostatin-28 (SST-28), an N-terminally elongated form of SST-14. SST-14 is a cyclic peptide with a length of 14 residues, containing a disulfide linkage between cysteines at positions 3 and 14. SST-28 is an N-terminal extension form (28 residues) of the same precursor that is proteolytically cleaved to generate SST-14. Although the two have similar activity, their respective potency and histological characteristics vary. For example, SST-14 displays more pronounced inhibition of glucagon and gastrin, while SST-28 displays more pronounced inhibition of growth hormone and insulin action. Both forms of somatostatin exert their respective biological functions through receptors on target cells and intracellular pathways. Five subtypes of somatostatin receptors (SSTR 1-5) have been recognized, with two spliced variants for SSTR2: SSTR2A and SSTR2B, with a different carboxyl terminus.

The beneficial effects of somatostatin in the treatment of certain hypersecretory endocrine disorders, and its anti-proliferation effect on tumors are well recognized. However, the half-life of somatostatin in vivo is only 2-3 minutes due to enzymatic degradation and endocytosis, limiting clinical utility of somatostatin. In the past decade, numerous stable somatostatin analogs have been developed. For example, octreotide and lanreotide are used in treatment of growth hormone (GH)-secreting adenomas and carcinoids. However, therapeutic limitations still exist due to altered binding affinity to SSTRs. As a result, there remains a need in the art for somatostatin constructs that achieve high in vivo half-life while maintaining a desirable binding affinity to SSTRs.

Albumin, the most abundant protein in the blood plasma, is produced in the liver as a monomeric protein of 67 kDa and responsible for 80% of the colloid osmotic pressure of plasma. Human granulocyte colony stimulating factor (G-CSF), human growth hormone (GH), human insulin, human interferon-a-2b (INF-2b), and interleukin-28B (IL-28B) fused with HSA were used effectively to construct long-acting therapeutic drug candidates. However, the comparative studies between HSA fusion proteins and the parent molecules in the biological and molecular mechanisms are less reported.

Chinese patent applications CN102391376A and CN102675467A, both hereby incorporated by reference, disclose somatostatin-albumin fusion proteins. However, there remains a need for further development of somatostatin-albumin fusion proteins.

### SUMMARY OF THE INVENTION

The present invention provides somatostatin-albumin fusion proteins and analogues thereof and methods of producing and using the same. Constructs prepared according to the invention include an albumin (or an analog thereof) moiety, a somatostatin moiety (SST-14, SST-28), and a spacer, such as a spacer or linker peptide, separating the two moieties.

A fusion protein according to the invention is also described as a polypeptide herein. The polypeptide according to the invention may optionally include, in certain embodiments, one or more non-naturally occurring amino acids or amino acid residues.

The somatostatin-albumin fusion proteins and analogues thereof broadly include a human SST peptide moiety, a linker or spacer, and a human albumen moiety. The SST peptide moiety can include analogues and derivatives thereof, that actively inhibit the activity of human growth hormone. Optionally, the SST peptide moiety is obtained from natural or synthetic sources. The albumin moiety is, e.g., human albumin and/or active fragments or subdomains thereof. The linker or spacer is selected to enhance the stability of the somatostatin-albumin fusion protein. More particularly, the somatostatin-albumin fusion proteins and analogues thereof have a structure as follows.

The invention provides for a fusion protein comprising:

an SST;

an L; and

an ALB,
wherein,
SST is a somatostatin, its analogue or derivative;
L is a spacer or a linker; and
ALB is an albumin, its analogue or variant.

Preferably, the inventive fusion protein is isolated and purified.

Optionally, the ALB component of the inventive fusion protein is mammalian serum albumin. In one embodiment, the mammalian serum albumin is SEQ ID NO: 25, or a sequence having at least 85% sequence identity thereto.

In other particular embodiments, the inventive fusion protein is selected from the group consisting of:

SST-(L)ₓ₁-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-SST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[SST-(L)ₓ₂]_{y1}-ALB-[(L)ₓ₃-SST]_{y2}-(L)ₓ₁-ALB-[(L)ₓ₄-SST]_{y3} (X);
wherein,
x1, x2, x3, x4, y1, y2, or y3 is independently zero or an integer selected from 1-10, or more particularly from 1-5, or an integer from 1-4, provided that there is at least one L present in the nucleotide sequence encoding an albumin-somatostatin fusion protein.

In alternative embodiments, the inventive fusion protein comprises an SST that is either naturally occurring or synthetically manufactured.

In a further embodiment, the SST of the inventive fusion protein comprises one or more tandem repeats of a sequence encoding SST-14 or SST-28, represented by SEQ ID NOS: 17 or 18, respectively, or a sequence having at least 85% identity to either of these sequences.

The SST moiety is optionally SST-14 or SST-28.

In a further embodiment, the fusion protein comprises an L that is either flexible or alpha helically structured polypeptide linker or spacer.

In a further embodiment, the fusion protein comprises an L that is a polypeptide having 2-100 amino acids. The linkers or spacers according to a further embodiment of the invention encompass peptides covalently linked to somatostatin on one terminal and to albumin on another terminal.

The terms “linker” or “spacer” are used interchangeably herein to refer to short amino acid sequences used to separate multiple domains in a single protein. Absence of linkers between two or more discrete domains in a protein may result in reduced or improper functionality of the protein domains e.g., a reduction in catalytic activity or binding affinity for a receptor/ligand, due to the steric hindrance. Linking protein domains in the chimeric proteins using an artificial linker can increase the space between the domains. Preferably, the linker or spacer is selected independently of the somatostatin and albumin.

The linker L is either a flexible or alpha helically structured polypeptide linker or spacer. In certain embodiments, L contains at least one GGGGS, A(EAAAK)₄A, (AP)n, wherein n is an integer selected from 10-34, (G)8, (G)5, or any combination thereof.

The albumin-somatostatin fusion constructs described herein may also include a signal peptide sequence (“SP”). Signal peptides are understood to refer to short amino acid sequences present at the N-terminus of a polypeptide that direct the cellular placement of a newly-synthesized protein. For example, signal peptides may lead to a protein being localized to a given intracellular region (e.g., the nucleus), inserted into a membrane (e.g., the cell membrane or the endoplasmic reticulum) or secreted from the cell. Besides directing localization, signal peptides may also be incorporated into recombinant proteins in order to improve stability, modify expression levels, and to aid in the proper folding of the recombinant proteins. The signal peptide sequence of the precursor protein is usually removed by signal peptidase in the host cell to produce a mature protein.

The albumin-somatostatin fusion constructs described herein may also include an affinity or purification tag as part of the polypeptide sequence to facilitate purification. Such tags are used as part of affinity chromatographic methods, e.g., high performance liquid chromatography (HPLC) in order to purify a protein sample from a crude biological source. Suitable purification tags include, but are not limited to: poly-histidine (e.g., His-6 or H6), glutathione-S-transferase (GST), maltose-binding protein (MBP), chitin binding protein (CBP), FLAG-tag (FLAG octapeptide). When it is necessary to remove the affinity tag from the fusion protein, specific enzymatic cleavage site can be introduced in the linker region. Enzymes commonly used for removal of affinity tags include, but are not limited to: factor Xa, entrokinase, thrombin, TEV protease, and rhinovirus 3C protease.

In a further embodiment, the invention provides a nucleotide sequence encoding a polypeptide comprising:

an SST;

an L; and

an ALB,
wherein,
SST is a somatostatin or its analogues or derivatives;
L is a spacer or a linker; and
ALB is an albumin or its analogues or variants.

In particular embodiments the inventive nucleotide encodes a polypeptide that is selected from the group consisting of,

SST-(L)ₓ₁-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-SST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[SST-(L)ₓ₂]_{y1}-ALB-[(L)ₓ₃-SST]_{y2}-(L)ₓ₁-ALB-[(L)ₓ₄-SST]_{y3} (X);
wherein,
each of x1, x2, x3, x4, y1, y2, or y3 is independently zero or an integer selected from 1-10, or more particularly from 1-5 or from 1-4, provided that there is at least one L present in the polypeptide.

In a further embodiment, the nucleotide sequence encodes a fusion protein wherein the SST comprises one or more tandem repeats of a sequence encoding SST-14 or SST-28, represented by SEQ ID NOS: 17 or 18, respectively, or a sequence having at least 85% identity to either of these sequences.

The invention is further contemplated to include an expression vector, e.g., a plasmid construct, a host cell comprising the expression vector, that is capable of expressing the inventive albumin-somatostatin fusion protein. The host cell can be a suitable bacterial host cell, a suitable mammalian host cell, a suitable plant host cell, or a suitable insect host cell.

The invention also provides for methods of treating a disease or disorder of endocrine release in a mammal, such as in a human subject, by administering an effective amount of a pharmaceutical composition comprising the inventive fusion protein, wherein the disease or disorder of endocrine release is a condition that responds to the administration of somatostatin.

For example, the disease or disorder is a cancer selected from the group consisting of breast cancer, colorectal cancer, liver cancer, endocrine cancer, neuroendocrine cancers, pancreatic cancer, prostate cancer, brain cancer, and lung cancer. In certain embodiments, the cancer expresses somatostatin receptor type 1, 2, 3, 4 or 5.

It should also be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes.

All numerical ranges should be understood to include each and every numerical point within the numerical range, and should be interpreted as reciting each and every numerical point individually. The endpoints of all ranges directed to the same component or property are inclusive, and intended to be independently combinable.

As used herein, the term “about” means within 10% of the reported numerical value, preferably within 5% of the reported numerical value.

The phrase “consisting essentially of” means that the composition or method may include additional ingredients and/or steps, but only if the additional ingredients and/or steps do not materially alter the basic and novel characteristics of the claimed composition or method.

The SST and albumin fusion proteins of present application provide advantages over natural SST of (a) higher in vivo stability, (b) higher binding affinity to SST receptors, (c) higher protein expression yield, and (d) better pharmacokinetic/pharmacodynamics behavior.

Before the present invention is described in detail below, it is to be understood that this invention is not limited to the particular methodology, protocols and reagents described herein, as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

### BRIEF DESCRIPTION OF THE FIGURES

FIG. 1 illustrates the pharmacokinetic profile of SST in rat.

The diamond (♦) labeled curve represents the data from Rat #6

The triangle (▴) labeled curve represents the data from Rat #7

The square (▪) labeled curve represents the data from Rat #8

The “Y” axis is Ln(Ct/C0) that represents the natural log of the measured plasma concentration of SST at time “t” (Ct) divided by the initial measured plasma concentration (C0) of SST

The “X” axis is plasma sampling time (‘t”) in hours.

Note that at certain time points, the detection of SST plasma concentration was below limits of quantitation

FIG. 2 illustrates bi-phasic pharmacokinetic profile of SST Fusion Protein in rat (Black dotted line distinguishes between α-Phase (0-0.5 hour) and β-Phase (0.75-4 hours).

The diamond (♦) labeled curve represents the data from Rat #1

The triangle (▴) labeled curve represents the data from Rat #2

The star (*) labeled curve represents the data from Rat #3

The square (▪) labeled curve represents the data from Rat #4

The “x” (x) labeled curve represents the data from Rat #5

The “Y” axis is Ln(Ct/C0) that represents the natural log of the measured plasma concentration of SST fusion protein at time “t” (Ct) divided by the initial measured plasma concentration (C0) of SST fusion protein

The “X” axis is plasma sampling time (‘t”) in hours.

### DETAILED DESCRIPTION OF THE INVENTION

The present invention encompasses somatostatin-albumin fusion proteins and analogues thereof and methods of producing and using the same. Constructs prepared according to the invention include an albumin (or an analog thereof) moiety, a somatostatin moiety (e.g., SST-14, SST-28), and a spacer separating the two moieties.

The somatostatin-albumin fusion proteins of the certain embodiment of the invention include variants of albumin including human serum albumin and/or derivatives of somatostatin. The spacers of another embodiment of the invention encompass peptides covalently linked to somatostatin on one terminal and albumin on another terminal. The spacers in other embodiments of the invention include peptide sequences having 2-100 amino acids.

In one embodiment, the present invention provides a fusion protein comprising:

an SST;

an L; and

an ALB,
wherein,
SST is a somatostatin or its analogues or derivatives;
L is a spacer or a linker;
ALB is an albumin or its analogues or variants.

In certain embodiments, the fusion protein of the present invention is selected from among formulas I-X, as follows.

SST-(L)ₓ₁-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-SST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[SST-(L)ₓ₂]_{y1}-ALB-[(L)ₓ₃-SST]_{y2}-(L)ₓ₁-ALB-[(L)ₓ₄-SST]_{y3} (X);
wherein,
each x1, x2, x3, x4, y1, y2, or y3 is independently zero or an integer selected from 1-10,
provided that there is at least one L present in the fusion protein.

In yet another embodiment, the present invention provides a nucleotide sequence encoding an albumin-somatostatin fusion protein comprising:

an SST;

an L; and

an ALB,
wherein,
SST is a somatostatin or its analogues or derivatives;
L is a spacer or a linker;
ALB is an albumin or its analogues or variants.

In certain embodiments, the nucleotide sequence of the present invention is selected to encode an albumin-somatostatin fusion protein from among,

SST-(L)ₓ₁-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-SST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[SST-(L)ₓ₂]_{y1}-ALB-[(L)ₓ₃-SST]_{y2}-(L)ₓ₁-ALB-[(L)ₓ₄-SST]_{y3} (X);
wherein,
each x1, x2, x3, x4, y1, y2, or y3 is independently zero or an integer selected from 1-10, 1-5 or 1-4, provided that there is at least one L present in the nucleotide sequence encoding an albumin-somatostatin fusion protein.

Another embodiment of the present invention provides a nucleotide sequence encoding an albumin-somatostatin fusion protein, wherein the spacer sequence consists of the sequence encoding the amino acid sequence represented by SEQ ID NO: 31 or -GGGGS-.

Another certain embodiment of the present invention provides a nucleotide sequence encoding an albumin-somatostatin fusion protein, wherein the second region (b) encodes a polypeptide having at least 85% sequence identity to SEQ ID NO: 19, albumin or a fragment thereof.

One embodiment of the present invention provides a nucleotide sequence encoding an albumin-somatostatin fusion protein, wherein the first region (a) encodes a polypeptide having at least 85% sequence identity to either SEQ ID NOS: 17 or 18, SST-14, SST-28, or a fragment thereof.

The present invention also provides a nucleotide sequence encoding an albumin-somatostatin fusion protein comprising:

(a) a first region comprising a nucleotide sequence containing one or more adjacent repeats of a sequence encoding a human somatostatin peptide;

(b) a second region comprising a nucleotide sequence encoding human serum albumin, or a fragment thereof;

(c) a spacer region comprising a nucleotide sequence encoding a polypeptide of 2-100 residues in length;

wherein the spacer region is present between the first region and the second region, or or between the first region and another first region;

wherein one or more adjacent repeats of a sequence encoding a human somatostatin peptide encodes either SST-14 or SST-28, as represented by SEQ ID NOS:17 and 18, respectively, or a sequence having at least 85% identity to either of these two sequences; or

wherein the spacer sequence consists of the sequence encoding the amino acid sequence represented by SEQ ID NO: 31 or GGGGS or by SEQ ID NO: 30 A(EAAAK)₄A; or

wherein the region (a) consists of one or more adjacent repeats of either SST-14 or of SST-28, as represented by SEQ ID NOS: 23 and 24, respectively, or a sequence having at least 85% identity to either of these two sequences.

Furthermore, the present invention provides a polypeptide sequence an albumin-somatostatin fusion protein comprising:

(a) a first region comprising a polypeptide sequence of a somatostatin peptide (which may be a human somatostatin peptide);

(b) a second region comprising a polypeptide sequence of serum albumin (which may be a human serum albumin), or a fragment thereof;

(c) a spacer region comprising a polypeptide of 2-100 residues in length.

The spacer region (c) may be present between region (a) and region (b) or between region (a) and region (a). In addition, the region (a) may comprise one or more tandem repeats of a sequence encoding SST-14 or SST-28, represented by SEQ ID NOS: 17 or 18, respectively, or sequence having 85% identity to either of these sequences.

Another embodiment of the present invention provides a plasmid construct expressing an albumin-somatostatin fusion protein with any of the fusion protein or polypeptide sequences described above.

Yet another embodiment of the present invention includes a bacterial cell transformed with the plasmid construct described above.

A further embodiment of the present invention includes an isolated and purified albumin-somatostatin fusion protein having the polypeptide sequence described above (e.g., a polypeptide sequence of an albumin-somatostatin fusion protein or the plasmid construct expressing such protein).

TABLE 1
A non-exclusive list of polypeptide sequences

| SEQ ID NO: | Description |
|---|---|
| SEQ ID NO: 1 | SST14-A(EAAAK)₄A-HSA-A(EAAAK)₄A-SST14 |
| SEQ ID NO: 2 | HSA-A(EAAAK)₄A-SST14 |
| SEQ ID NO: 3 | His6-GGS-HSA-GGGGS-SST14-HSA |
| SEQ ID NO: 4 | His6-GGS-HSA-GGGGS-(SST14-GGGGS)₂-HSA |
| SEQ ID NO: 5 | HSA-GGGGS-(SST14-GGGGS)₂-HSA |
| SEQ ID NO: 6 | Linker GGGGGGGG |
| SEQ ID NO: 7 | SST14-(GGGGS)₃-HSA |
| SEQ ID NO: 8 | SST14-A(EAAAK)₄A-HSA |
| SEQ ID NO: 9 | His6-GGS-HSA-GGGGS-SST14 |
| SEQ lD NO: 10 | SST14-GGGGS-HSA-GGS-His6 |
| SEQ lD NO: 11 | HSA-GGGGS-SST14 |
| SEQ lD NO: 12 | SST14-GGGGS-HSA |
| SEQ lD NO: 13 | (SST14-GGGGS)₂-HSA |
| SEQ lD NO: 14 | (SST14-GGGGS)₄-HSA |
| SEQ lD NO: 15 | HSA-(GGGGS)₃-SST14 |
| SEQ lD NO: 16 | HSA-(GGGGS)₆-SST14 |
| SEQ ID NO: 17 | Somatostatin-14 (SST-14) |
| SEQ ID NO: 18 | Somatostatin-28 (SST-28) |
| SEQ ID NO: 19 | Human Serum Albumin (HSA) |
| SEQ lD NO: 20 | MDMRVPAQLLGLLLLWLRGARC (Signal Peptide) |
| SEQ lD NO: 21 | Linker APAPAPAPAPAPAPAPAPAP |
| SEQ lD NO: 22 | Linker APAPAPAPAPAPAPAPAPAPAPAPAPAPAPAPAPAPAPAP |
| SEQ lD NO: 30 | A(EAAAK)₄A peptide |
| SEQ lD NO: 31 | GGGGS peptide |
| SEQ ID NO: 32 | Linker GGGGSLVPRGSGGGGS |
| SEQ lD NO: 33 | Linker GSGSGS |
| SEQ lD NO: 34 | Linker GGGGSLVPRGSGGGG |
| SEQ ID NO: 35 | Linker GGGGSLVPRGSGGGGS |
| SEQ ID NO: 36 | Linker GGSGGHMGSGG |
| SEQ lD NO: 37 | Linker GGSGGSGGSGG |
| SEQ ID NO: 38 | Linker GGSGGHMGSGG |
| SEQ lD NO: 39 | Linker GGSGG |
| SEQ ID NO: 40 | Linker GGGGSLVPRGSGGGGS |
| SEQ ID NO: 41 | Linker GGSGGGGG |
| SEQ lD NO: 42 | Linker GSGSGSGS |
| SEQ ID NO: 43 | Linker GGGSEGGGSEGGGSEGGG |
| SEQ ID NO: 44 | Linker AAGAATAA |
| SEQ ID NO: 45 | Linker GGGGG |
| SEQ ID NO: 46 | Linker GGSSG |
| SEQ ID NO: 47 | Linker GSGGGTGGGSG |
| SEQ ID NO: 48 | Linker GSGSGSGSGGSGGSGGSGGSGGSGGS |

For the fusion proteins, e.g., SEQ ID NOs: 1-5, 7-10 and 13-16, it should be noted that these are encoded as pro-proteins with a 22 residue signal peptide (SEQ ID NO: 20).

### Somatostatin-Albumin Fusion Proteins

The invention encompasses polypeptide constructs wherein the somatostatin moiety is encoded by a nucleotide having at least 85% sequence identity to the nucleotide sequence of endogenous human SST-14 or SST-28 (SEQ ID Nos: 23 and 24, respectively).

The invention also encompasses polypeptide constructs wherein the human serum albumin moiety is encoded by a nucleotide having at least 85% sequence identity to the nucleotide sequence of endogenous human serum albumin (SEQ ID NO: 25). The invention further encompasses polypeptide constructs wherein the human serum albumin moiety is a fragment of the endogenous human serum albumin protein, e.g., where it is encoded by a nucleotide consisting of a subsequence of SEQ ID NO: 25. For example, the human serum albumin fragment optionally includes one or more of the three human serum albumin globular domains, each of which contains two subdomains, denominated subdomain IA, IB, IIA, IIB, IIIA, and IIIB (Dockal, 1999, The Journal Of Biological Chemistry, 274(41): 29303-29310).

The invention also encompasses polypeptide constructs wherein the somatostatin moiety has a polypeptide sequence at least 85% sequence identity, preferably at least 90% to the polypeptide sequence of endogenous SST-14 or SST-28 (SEQ ID NOs:17 and 18, respectively).

The invention also encompasses polypeptide constructs wherein the human serum albumin moiety has a polypeptide sequence at least 85% sequence identity to the polypeptide sequence of mature human serum albumin (SEQ ID NO: 19).

The invention also encompasses a fusion protein comprising a signal peptide, a purification tag (His-6), a first linker, a human serum albumin moiety, a second linker and a somatostatin moiety. In one embodiment, the fusion protein is a polypeptide is represented by SEQ ID NO: 9 or a sequence having 85% sequence identity to the same.

The invention also encompasses a fusion protein comprising a somatostatin moiety, a first linker, a human serum albumin moiety, a second linker, a somatostatin moiety and a purification tag (His-6). In one embodiment, the fusion protein is a polypeptide is represented by SEQ ID NO: 10 or a sequence having 85% sequence identity to the same.

The invention also encompasses a nucleotide sequence (SEQ ID NO: 11) encoding a fusion protein comprising an N-terminal human serum albumin moiety and a C-terminal somatostatin moiety separated by a peptide spacer. The invention further encompasses nucleotide sequences encoding an albumin-somatostatin fusion construct which have 85% sequence identity to SEQ ID NO: 11.

The invention also encompasses a nucleotide sequence (SEQ ID NO: 12) encoding a fusion protein comprising an N-terminal somatostatin moiety and a C-terminal human serum albumin moiety separated by a peptide spacer. The invention further encompasses nucleotide sequences encoding an albumin-somatostatin fusion construct which have 85% sequence identity to SEQ ID NO: 12.

The invention also encompasses polypeptide constructs wherein the somatostatin moiety comprises two or more copies of the SST-14 or SST-28 sequence arranged in tandem, i.e., “(SST-14)₂” or “(SST-14)₃” or “(SST-28)₂” or “(SST-28)₃”, respectively. Optionally, a linker sequence is included between the two or more tandem somatostatin moieties, and/or a signal peptide sequence is included at the N-terminus of the fusion protein.

The invention also encompasses polypeptide constructs wherein the somatostatin moiety comprises two or more copies of the SST-14 sequence arranged in a way that at least one copy of the SST14 is linked on both sides of albumin, respectively. Optionally, a linker sequence is included between the two or more tandem somatostatin moieties and between somatostatin and albumin, and/or a signal peptide sequence is included at the N-terminus of the fusion protein. For example, the polypeptide construct may include a signal peptide, two SST-14 moieties separated by a spacer, a second spacer, and an HSA moiety as represented. Optionally, the construct omits the N-terminal signal peptide.

The invention also encompasses polypeptide constructs wherein the somatostatin moiety comprises two or three copies of the SST-28 sequence arranged in tandem, i.e., “(SST-28)₂” or “(SST-28)₃”, respectively. Optionally, a linker sequence is included between the two or more tandem somatostatin moieties.

The invention also encompasses polypeptide constructs comprising any of the albumin-somatostatin fusion proteins described in the preceding paragraphs, where the albumin-somatostatin fusion protein has an in vivo half-life longer than the endogenous SST-14 or SST-28 peptides.

The invention also encompasses polypeptide constructs comprising any of the albumin-somatostatin fusion proteins described in the preceding paragraphs, wherein the albumin-somatostatin fusion protein has an approximately equal or a greater binding affinity for a somatostatin receptor compared to endogenous SST-14 or SST-28.

The invention also encompasses albumin-somatostatin fusion proteins comprising an N-terminal albumin moiety as represented by SEQ ID NO: 15, SEQ ID NO: 16, and SEQ ID NO: 2, an internal SST moiety and a C-terminal Albumin moiety as represented by SEQ ID NO: 7 and SEQ ID NO: 8. Optionally, the N-terminus may further include a signal peptide. Optionally, one or more of the albumin and SST domains may each be separated by an independently selected linker sequence as represented by SEQ ID NO: 1.

In some embodiments, the SST moiety may comprise a pair or plurality of tandem SST sequences, e.g., (SST-14)₂ or (SST-28)₃, with or without intervening spacing sequences between the two or more tandem SST repeats. Optionally, one or more purification tag sequences may be included in the sequence between two moieties or at the N or C-terminus in order to assist with purification of the fusion protein. An alternative embodiment includes a pair of SST-14 moieties separated by a spacer, as represented by SEQ ID NO: 4. A further embodiment may omit the purification tag (e.g., His6) as shown by the polypeptide sequence represented by SEQ ID NO: 5.

### Somatostatin

The somatostatin for use with the present invention may be any somatostatin, its analogue or derivative. It may be a human somatostatin, any other isolated or naturally occurring somatostatin. The SST moiety can be an analogue such as octreotide, lanreotide, pasireotide, seglitide, or vapreotide.

The invention also encompasses polypeptide constructs wherein the somatostatin moiety comprises a somatostatin analog. Preferably, such an analog is suitable for expression, as part of a fusion protein, in a recombinant host cell. It is understood that a suitable somatostatin analog sequence may be used in place of the SST-14 or SST-28 sequences included in any of the examples disclosed herein.

The invention also encompasses polypeptide constructs wherein the somatostatin moiety comprises two or more tandem repeats of a somatostatin polypeptide sequence e.g., SST-14 or SST-28; SEQ ID NOS: 17 and 18, respectively. Each of the repeated somatostatin polypeptide sequences may be a polypeptide sequence having at least 85% sequence identity to SST-14 or SST-28. These repeated variant sequences are independently selected, i.e., in some embodiments the repeats are identical, whereas in other embodiments they are unique.

### Albumin

The albumin for use with the present invention may be any albumin, its analogue or variant. The albumin may be human serum albumin, or any other isolated or naturally occurring albumin.

The invention also encompasses polypeptide constructs wherein the human serum albumin moiety comprises a polypeptide sequence variant with alternative arrangement or number of disulfide bonds due to the presence of additional or fewer cysteine residues than the natural form (SEQ ID NO: 25).

### Spacer or Linker

As described earlier, a spacer or linker can be used with the present invention. The spacer or linker may be independent of the somatostatin or albumin.

The invention also encompasses polypeptide constructs wherein the peptide spacer of alternatively referred to as a linker, consists of a polypeptide sequence of from about 2 to about 100 amino acid residues in length. The invention further encompasses polypeptide constructs wherein the peptide spacer is from about 2 to about 50 amino acid residues in length, preferably from about 2 to about from 30, or more preferably from about 3 to about 20 amino acid residues in length.

The invention also encompasses polypeptide constructs wherein the peptide spacer (alternatively referred to as a linker) has the polypeptide sequence “GGGGS” (SEQ ID NO: 31). Polypeptides rich in Gly, Ser or Thr offer special advantages include, but not limited to: (i) rotational freedom of the polypeptide backbone, so that the adjacent domains are free to move; (ii) enhanced solubility; (iii) resistance to proteolysis. In addition, many natural linkers exhibited alpha-helical structures. The alpha-helical structure is more rigid and stable than Gly rich linker. An empirical rigid linker with the sequence of A(EAAAK)₄A (SEQ ID NO: 30) can be used to separate functional domains. In addition to the role of linking protein domains together, artificial linkers may offer other advantages to the production of fusion proteins, such as improving biological activity, increasing protein expression, and achieving desirable pharmacokinetic profiles.

TABLE 2
A non-exhaustive list of linker sequences that
may be used in the fusion protein
constructs of the present invention.

GGGGSLVPRGSGGGGS
(SEQ ID NO: 32)
GSGSGS
(SEQ ID NO: 33)
GGGGSLVPRGSGGGG (thrombin proteolytic site is
underlined)
(SEQ ID NO: 34)
GGGGSLVPRGSGGGGS (thrombin proteolytic site is
underlined)
(SEQ ID NO: 35)
GGSGGHMGSGG
(SEQ ID NO: 36)
GGSGGSGGSGG
(SEQ ID NO: 37)
GGSGGHMGSGG
(SEQ ID NO: 38)
GGSGG
(SEQ ID NO: 39)
GGGGSLVPRGSGGGGS (thrombin proteolytic site is
underlined)
(SEQ ID NO: 40)
GGSGGGGG
(SEQ ID NO: 41)
GSGSGSGS
(SEQ ID NO: 42)
GGGSEGGGSEGGGSEGGG
(SEQ ID NO: 43)
AAGAATAA
(SEQ ID NO: 44)
GGGGG
(SEQ ID NO: 45)
GGSSG
(SEQ ID NO: 46)
GSGGGTGGGSG
(SEQ ID NO: 47)
GT
GSGSGSGSGGSGGSGGSGGSGGSGGS
(SEQ ID NO: 48)
GGS
GGGGGGGG
(SEQ ID NO: 6)
A(EAAAK)₄A
(SEQ ID NO: 20)
APAPAPAPAPAPAPAPAPAP
(SEQ ID NO: 21)
APAPAPAPAPAPAPAPAPAPAPAPAPAPAPAPAPAPAPAP
(SEQ ID NO: 22)

### Preparation of Somatostatin-Albumin Fusion Protein

An embodiment of the invention provides a method for preparation of the somatostatin-albumin fusion protein. In one embodiment, the somatostatin-albumin fusion protein of the invention is prepared by expressing a vector containing the encoding gene and introducing the vector into a suitable host cell. For example, the fusion protein is obtained by expression of a suitable vector in a host such as yeast. In one embodiment, Pichia pastoris GS 115 may be used as a suitable expression host, and the vector is pPIC9K. In particular, mammalian cell lines such as CHO or HEK293 can be used as an expression host.

The invention also encompasses plasmid constructs capable of expressing an albumin somatostatin fusion protein comprising a nucleotide sequence encoding a somatostatin albumin fusion protein as described in any of the preceding paragraphs. For example, suitable plasmid constructs include, but are not limited to, the pcDNA3.1 vector represented by SEQ ID NO: 26 with a DNA sequence encoding any of the albumin-somatostatin fusion proteins disclosed herein ligated into the multiple cloning site of this vector. Other suitable protein expression vectors known in the art may be selected based upon the expression host (e.g., an expression vector with a mammalian promoter system would be suitable for expression in a human cell line whereas a yeast or bacterial expression plasmid would be selected if expression in either of these organisms was desired.

The invention also encompasses a bacterial or yeast protein expression system comprising a bacterial or yeast cell transformed with a plasmid construct comprising a nucleotide sequence that encodes a somatostatin albumin fusion protein, as described in any of the preceding paragraphs. Suitable bacterial strains include, for example, Escherichia coli. Suitable yeast strains include, for example, Pichia pastoris. An exemplary plasmid construct includes pPIC9K (Invitrogen) as represented by SEQ ID NO: 27, with a nucleotide sequence encoding any of the albumin-somatostatin fusion proteins described herein incorporated into the multiple cloning site of the vector.

The invention also encompasses isolated and purified fusion somatostatin fusion proteins having a polypeptide sequences as described in any of the preceding paragraphs.

TABLE 3
A list of nucleotide sequences in certain embodiments of the invention

| SEQ ID NO: | Nucleotide Sequence Encodes the following: | Description |
|---|---|---|
| SEQ ID NO: 23 | SST14 | Somatostatin-14 |
| | | (SST-14) |
| SEQ ID NO: 24 | SST28 | Somatostatin-28 |
| | | (SST-28) |
| SEQ ID NO: 25 | Human Serum Albumin mature form | Human Serum Albumin |
| | | (HSA) |
| SEQ ID NO: 26 | pcDNA3.1(+) Vector | pcDNA3.1(+) Vector |
| | | mammalian expression |
| | | vector |
| SEQ ID NO: 27 | pPIC9K Vector | yeast expression vector |
| SEQ ID NO: 28 | GGGGS | GGGGS Linker |
| SEQ ID NO: 29 | A(EAAAK)₄A | alpha-helical linker |

When the SST is a somatostatin analogue, an alternative method known in the field can be employed to prepare the conjugate.

### Utility of Somatostatin-Albumin Fusion Protein

The fusion protein of the present invention can be used to treat conditions for which somatostatin is art-known to be employed. As such, the invention also encompasses methods of treating cancer in a human subject by administering an isolated and purified albumin-somatostatin fusion protein as described in any of the preceding paragraphs, wherein the cancer is any cancer known to respond to somatostatin treatment, e.g., selected from breast cancer, colorectal cancer, liver cancer, lung cancer, endocrine cancer, neuroendocrine cancers, pancreatic cancer and prostate cancer.

The invention also encompasses methods of treating cancer in a human subject by administering a composition containing the fusion protein of the present invention, such as an isolated and purified albumin-somatostatin fusion protein as described in any of the preceding paragraphs. The composition can also include a suitable carrier.

Eleven SST14-Albumin fusion protein constructs with various linker sequences were designed. Eight of these constructs were made into a fusion gene within a plasmid and produced by HEK 293 transient expression at 100 mL scale. The proteins were collected from the culture media, purified through albumin-based affinity purification, and dialyzed to a storage buffer. These fusion proteins were evaluated for their binding affinity to SSTR2 receptor, and also for cell-based activity in inhibiting cAMP production in a SSTR2-overexpression CHO-K1 cell line. The results of these studies indicated that the length and type of linkers significantly affected the SSTR2 receptor binding affinity, the in-vitro cell-based functional activity, and the fusion protein production yield.

SST-Albumin fusion protein of this invention exhibited a significantly longer serum half-life and/or improved pharmacokinetic profile in solution or in a pharmaceutical composition in vitro and/or in vivo compared to the corresponding unfused, free SST molecules. The stability of free SST and SST fusion protein was compared in in vitro rat plasma. When incubated in freshly prepared rat plasma at 37° C., free SST and SST fusion protein exhibited degradation half-lives of 33 minutes and 5.5 hours, respectively (Table 4).

In vivo pharmacokinetic profiles were also generated to demonstrate the improved stability of SST fusion protein relative to free SST. Rats administered intravenously with SST showed a calculated T_{1/2} of 3.5 minutes. On the other hand, rats administered intravenously with SST fusion protein exhibited a bi-phasic pharmacokinetic profile, where the α-phase T_{1/2} was 1.01 hour and the β-phase T_{1/2} was 6.14 hour. The calculated half-life of SST fusion protein is significantly longer than the calculated T_{1/2} of free SST in rat (3.5 minutes) and the reported plasma T_{1/2} of free SST in rat (<1 minute; Reference #1) (Table 4).

TABLE 4
Calculated Half-life of Free SST and SST Fusion Protein for In Vitro Plasma
Stability and In Vivo Rat Model

| T_{1/2} | In Vitro Plasma Stability | | In Vivo Rat Pharmacokinetic | |
|---|---|---|---|---|
| Free SST | 33 | minutes | 3.5 ± 1.0 minutes | |
| | | | less than 1 minutes (Ref. #1) | |
| SST Fusion Protein | 5.5 | hour | α phase (0-0.5 hr) | 1.01 ± 0.60 hr |
| | | | β phase (0.75-4 hr) | 6.14 ± 1.27 hr |

Reference #1: Yogesh C. Patel and Thomas Wheatley. In Vivo and in Vitro Plasma Disappearance and Metabolism of Somatostatin-28 and Somatostatin-14 in the Rat. Endocrinology. Vol. 112, No. 1 (1992), pages 220-225.

### EXAMPLES

Selected embodiments of the invention will be described in further detail with reference to the following experimental and comparative examples. These examples are for illustrative purposes only and are not intended to limit the scope of the invention.

### Example 1

### Expression in Mammalian Systems

### Example 1-1

### Recombinant Gene Synthesis

Eight constructs corresponding to the fusion proteins listed in Table 5 were prepared. First, the gene sequence coding each fusion protein was de novo synthesized and subsequently inserted into the pcDNA3.1 vector.

### Example 1-2

### Plasmid Generation

Maxi-prep or Mega-prep was used to generate ˜20 mg of each DNA

### Example 1-3

### Transfection and Protein Production

(A) Suspension Cell Method

FreeStyle™ 293-F Cells were seeded at 0.55-0.6×10⁶ cells/mL in a flask. After about 24 hours, the cells were seeded in a shake flask at 1.1-1.2×10⁶ cells/mL. DNA was prepared at 500 μg DNA/80 mL in a FreeStyle medium. Polyethylenimine (PEI) was prepared at 1.8 mL PEI per 80 mL in a FreeStyle medium. DNA was mixed in the FreeStyle medium, and the effective amount of PEI was added to the DNA solution, and the mixture is vortexed incubated for about 15 minutes at room temperature to form a DNA-PEI complex. An 80 mL of the incubated DNA-PEI complex is added to a cell culture. About 3 hours later, TC Yeastolate feed (BD) is added to have the final concentration of 4 gram/liter of culture. After about 7-8 days, the medium is harvested by centrifugation.

(B) Adherent Cell Method

About 24 hours before transfection, HEK293 cells were seeded to 50-90% confluency in a flask, and complete medium is added. After about 24 hours, cells were washed followed by adding basal medium.

DNA and PEI solutions were prepared by adding DNA to a serum free medium. The PEI solution was added to the DNA solution and incubated for 15 minutes to form DNA-PEI complex at room temperature.

The PEI/DNA mixture was added to cells, and the mixture incubated for about 4-6 hours at 37° C. The medium was removed and fresh medium with Glutamine and serum was added, followed by incubating at 37° C. for 4 days.

The medium was harvested after about 4 days, by centrifuging to collect the supernatant. The precipitate was replenished with fresh medium with L-Glutamine for another 3-day incubation to repeat the harvesting process.

### Example 1-4

### Protein Concentration, Ni-NTA Purification and Buffer Exchange

The collected medium was concentrated by TFF system (Millipore) to a certain volume depending on purification methods (either continuous chromatography or manual batch purification).

The concentrated proteins was incubated with fresh Ni-NTA resin at about 4° C. in binding buffer and washed with wash buffer using either chromatography or batch system. The protein was eluted with elute buffer and fractions were collected and concentrated to recover the purified protein. The protein can be further purified using size exclusion chromatography purification.

The buffer of the final eluate can be exchanged by dialysis to a desired buffer.

### Example 2

### Yields of Several SST-Albumin Fusion Proteins

The SST-HSA fusion proteins were all expressed in soluble form with high yield. The length or the nature of the linkers can affect the protein yield and solubility of the fusion proteins. The results indicated that the production yield slightly decreased as the fusion protein constructs became longer and more complex. However, all the constructs exhibited yield for scale up production.

TABLE 5
SST14-HSA fusion protein expression yield

| | | Total | | Production |
|---|---|---|---|---|
| | | amino | MW | Yield |
| Sequence ID | Design | acids | (kDa) | (g/L) |
| SEQ ID NO: 1 | SST14-A(EAAAK)₄A-HSA- | 657 | 73.8364 | 0.26 |
| | A(EAAAK)₄A-SST14 | | | |
| SEQ ID NO: 2 | HSA A(EAAAK)₄A-SST14 | 621 | 70.1543 | 0.27 |
| SEQ ID NO: 7 | SST14-(GGGGS)₃-HSA | 614 | 69.112 | 0.33 |
| SEQ ID NO: 8 | SST14-A(EAAAK)₄A-HSA | 621 | 70.1543 | 0.25 |
| SEQ ID NO: 9 | H6-GGGGS-HSA-GGGGS-SST14 | 613 | 69.4874 | 0.30 |
| SEQ ID NO: 10 | SST14-GGGGS-HSA-GGGGS-H6 | 613 | 69.4874 | 0.41 |
| SEQ ID NO: 15 | HSA-(GGGGS)3-SST14 | 614 | 69.112 | 0.28 |
| SEQ ID NO: 16 | HSA-(GGGGS)6-SST14 | 629 | 70.1119 | 0.29 |

### Example 3

### Binding Affinity of Several SST-Albumin Fusion Proteins

This assay measures binding of [¹²⁵I]Somatostatin to human somatostatin sst2 receptors. CHO-K₁ cells stably transfected with a plasmid encoding the human somatostatin sst2 receptor are used to prepare membranes in modified HEPES pH 7.4 buffer using standard techniques. A 0.1 mg aliquot of membrane is incubated with 0.03 nM [¹²⁵I]Somatostatin and tested fusion proteins for 240 minutes at 25° C. Non-specific binding is estimated in the presence of 1 μM Somatostatin. Membranes are filtered and washed 3 times and the filters are counted to determine [¹²⁵I]Somatostatin specifically bound.

The competitive binding study ¹²⁵I-Tyr-somatostatin versus the fusion proteins demonstrated the following results. The efficiency of the inhibition varied depending on the construct of the fusion proteins. The fusion protein construct (SEQ ID NO: 1) with two alpha-helical linker, A(EAAAK)₄A, showed 100% inhibition of the somatostatin and its receptor interaction. The SEQ ID NO: 1 construct has two somatostatin moiety on both N and C terminal sides of human serum albumin. The smaller construct with one somatostatin on the C terminal side of human serum albumin linked by the same alpha-helical linker (SEQ ID NO: 2) showed 96% inhibition. The same construct with the more flexible GGGGS linker showed lower inhibition of 82-85% depending on the length. The length of GGGGS linkers also affected the inhibition. The construct with five amino acid GGGGS linker (SEQ ID NO: 9 and SEQ ID NO: 8) showed 57-59% inhibition whereas the constructs with 15 amino acid (SEQ ID NO: 15) or 30 amino acid GGGGS linkers (SEQ ID NO: 16) showed over 80%, suggesting that longer than five amino acid GGGGS would be more advantageous to SST function. A more rigid A(EAAAK)₄A (a-helical) linker would be more efficient in binding than flexible GGGGS linker. A multiple SST can increase the effective concentration of the ligand for SST receptor binding. The position of Histidine purification tag may not affect the binding. Changing the orientation or position of albumin in the fusion protein may further increase the efficiency of the protein binding.

TABLE 6
Inhibition of ¹²⁵I-Tyr¹-somatostatin binding on SSTR2 by the fusion proteins

| | | Inhibition % | IC₅₀ |
|---|---|---|---|
| Sequence ID | Construct | at 0.1 μM | (nM) |
| SEQ ID NO: 1 | SST14-A(EAAAK)₄A-Albumin-A(EAAAK)₄A-SST14 | 100 | 2.38 |
| SEQ ID NO: 2 | Albumin-A(EAAAK)₄A-SST14 | 96 | 9.41 |
| SEQ ID NO: 7 | SST14-(GGGGS)₃-Albumin | 70 | |
| SEQ ID NO: 8 | SST14-A(EAAAK)₄A-Albumin | 79 | |
| SEQ ID NO: 9 | His6-GGS-Albumin-GGGGS-SST14 | 59 | |
| SEQ ID NO: 10 | SST14-GGGGS-Albumin-His6 | 57 | |
| SEQ ID NO: 15 | Albumin-(GGGGS)₃-SST14 | 85 | 33 |
| SEQ ID NO: 16 | Albumin-(GGGGS)₆-SST14 | 82 | |
| SEQ ID NO: 17 | SST-14 | | 0.0069 |

### Example 4

### Inhibition of Several SST-Albumin Fusion Proteins to Camp Accumulation in SSTR2-Expressing Cells

Human recombinant somatostatin sst2a receptors expressed in CHO-K1 cells were used. Test compound and/or vehicle was incubated with the cells (2×10⁵ cells/mL) in incubation buffer for 20 minutes at 37° C. Test compound-induced decrease of cAMP by 50 percent or more (50%) relative to the 10 nM Octreotide response indicated sst2a receptor agonist activity.

The inhibition of the accumulation of cAMP was observed in SST receptor type 2 expressing CHO-K1 cells. The value of EC₅₀ was 260 nM. The constructs with longer linkers (SEQ ID NOS: 1, 15, and 2) exhibited lower EC₅₀ values, which coincided with the binding assay data. The alpha-helical linker appeared to be more efficient in the inhibition of cAMP production, when Albumin-(GGGGS)₃-SST14 and Albumin-(GGGGS)₃-SST14 EC₅₀ values were compared.

TABLE 7
EC50 value for the inhibition of cAMP production

| | | EC₅₀ values of the |
|---|---|---|
| | | inhibition of cAMP |
| Sequence ID | Construct | production (nM) |
| SEQ ID NO: 1 | SST14-A(EAAAK)₄A-Albumin-A(EAAAK)₄A-SST14 | 5.14 |
| SEQ ID NO: 2 | Albumin-A(EAAAK)₄A-SST14 | 17.6 |
| SEQ ID NO: 9 | H6-GGGGS-Albumin-GGGGS-SST14 | 260 |
| SEQ ID NO: 15 | Albumin-(GGGGS)₃-SST14 | 23 |
| | Octreotide | 0.041 |

### Example 5

### Determination of Stability of Free SST and SST Fusion Protein in Rat Plasma

Improved stability of SST fusion protein in rat plasma was proven using ELISA. The test results showed that SST fusion protein (SEQ ID NO: 1) exhibited a degradation half-life of 5.5 hours as opposed to free SST, which showed less than 33 minute half-life when incubated in rat plasma at 37° C. (Table 4).

### Example 5-1

### Preparation of Sample

500 pg/mL of free SST or with 750 ng/mL of SST fusion protein (SEQ ID NO: 1) were incubated in pooled rat plasma for 1 minute, 2 minute, 5 minute, 20 minute, 60 minute, 80 minute, 100 minute, 120 minute, 150 minute, and 180 minute. The blood samples at each time point were incubated in triplicate. All the blood samples were centrifuged at 5500 rpm for 10 minutes to obtain plasma samples for analysis. Pooled rat plasma was used as blank for background measurement. All samples were analyzed in duplicate.

All plasma samples with SST fusion protein were diluted 15-fold with pooled rat plasma. For example, to a 10 μL plasma sample with SST fusion protein was added 140 μL pooled rat plasma.

### Example 5-2

### Preparation of Free SST and SST Fusion Protein Standards

(1). Preparation of Free SST standard Standard Curve was Prepared by the Following Procedure. Duplicate Standard Points

were prepared by serially diluting Free SST Stock (1 mg/mL) with diluent buffer to produce 5, 2.5, 1.25, 0.625, 0.313, 0.156, 0.078 and 0.039 ng/mL solutions.

(2). Preparation of SST Fusion Protein Standard

Standard curve was prepared by the following procedure. Duplicate standard points were prepared by serially diluting SST fusion protein Stock (2.42 mg/mL) with diluent buffer to produce 225, 112.5, 56.2, 28.1, 14.1, 7.03, 3.51 and 1.76 ng/mL solutions.

### Example 5-3

### ELISA Assay Procedure

1) All kit components were maintained at room temperature (20-25° C.) before analysis.
2) 50 μL/well of standard, sample, or positive control solution was added to the kit. Then, 25 μL/well of primary antibody was added into each well except the Blank well. At last, 25 μL/well biotinylated peptide was added into each well except the Blank well. The immunoplate was incubated for 2 hours at room temperature with shaking at 300-400 rpm. The wells were emptied and washed three times with 300 μL washing solution. After the last wash, the wells were emptied by tapping the strip on an absorbent tissue.
3) The contents of the wells were discarded and each well was washed with 300 μL of 1×EIA assay buffer, discard the buffer was, invert and blot dry plate. Repeat 4 times.
4) Add 100 μL of SA-HRP solution into each well. Incubate the immunoplate for 1 hour at room temperature with shaking at 300-400 rpm.
5) Wash and blot dry the immunoplate 4 times with 1×EIA assay buffer as described above in step 3.
6) Add 100 μL of TMB substrate solution into each well. Cover the immunoplate to protect from light. Incubate the immunoplate for 1 hour at room temperature with shaking at 300-400 rpm.
7) Add 100 μL 2N HCl into each well to stop the reaction. The color in the well should change from blue to yellow. If the color change does not appear to be uniform, gently tap the plate to ensure thorough mixing. Proceed to the next step within 20 minutes.
8) Load the immunoplate onto Plate Reader. Read absorbance O.D. at 450 nm.

### Example 6

### Determination of In Vivo Pharmacokinetic Profile of Free SST and SST Fusion Protein in Rat

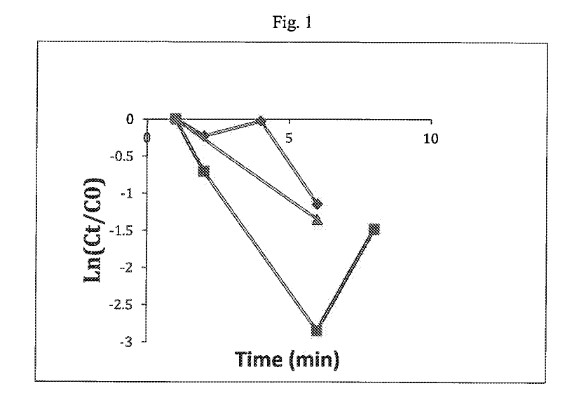

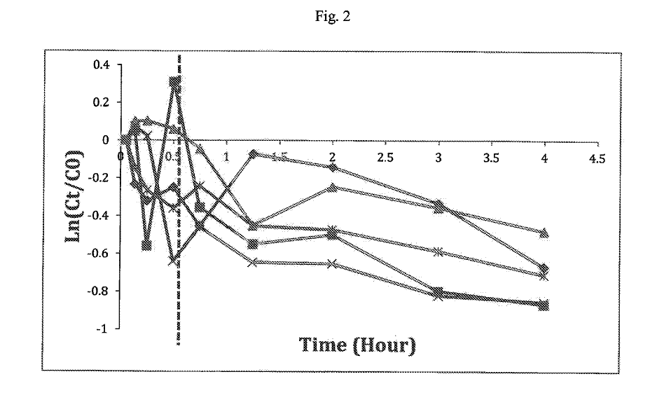

SST and SST fusion protein (SEQ ID NO: 1) were administered at 0.02 and 27.1 mg/kg, respectively, doses via tail vein injection to three and five, respectively, male Sprague Dawley rats to determine the pharmacokinetic profiles and parameters of SST and SST fusion protein, respectively (Table 8 and Table 9). The animals were fasted overnight with free access to water prior to injection, and no negative clinical signs were observed afterwards. SST exhibited rapid pharmacokinetic profile in each of the rats administered with SST (FIG. 1), and the calculated T_{1/2} was 3.5 minutes (Table 4). SST fusion protein exhibited a bi-phasic pharmacokinetic profile in each of the rats administered with SST fusion protein (FIG. 2), where the average α-phase T_{1/2} (0-0.5 hour) and β-phase T_{1/2} (0.75-4 hours) was calculated as 1.01 hour and 6.14 hour, respectively (Table 4). The calculated half-life of SST fusion protein was significantly longer than the calculated plasma T_{1/2} of free SST (3.5 minutes) and reported plasma T_{1/2} of free SST in rat (<1 minute; Reference #1) (Table 4). This set of results indicated the SST fusion protein significantly improves the stability and prolongs the half-life of SST in vivo.

### Example 6-1

### Preparation of Sample

The rat was restrained manually at the designated time points, approximately 300 μL of blood sample was collected via jugular vein into EDTA-K2 tubes and subsequently centrifuged at 4° C. and 1500 g for 10 min to obtain plasma samples.

### Example 6-2

### Preparation of SST and SST Fusion Protein Standards

(1). Preparation of Free SST Standard

Standard curve was prepared by the following procedure. Duplicate standard points were prepared by serially diluting Free SST Stock (1 mg/mL) with diluent buffer to produce 5, 2.5, 1.25, 0.625, 0.313, 0.156, 0.078 and 0.039 ng/mL solutions.

(2). Preparation of SST Fusion Protein Standard

Standard curve was prepared by the following procedure. Duplicate standard points were prepared by serially diluting SST fusion protein (2.42 mg/mL) with pooled rat plasma to produce 225, 112.5, 56.2, 28.1, 14.1, 7.03, 3.51 and 1.76 ng/mL solutions.

### Example 6-3

### ELISA Assay Procedure

ELISA assay has been performed as described in Example 5-3.

TABLE 8
Plasma SST and SST Fusion Protein Concentration after IV injection to Rat

| SST Dose | Dose | Sampling Time | Mean | Standard |
|---|---|---|---|---|
| (mg/kg) | Route | (min) | (ng/mL) | Deviation |
| 0.02 | IV | 0 | 0 | NA |
| | | 1 | 7.13 | 5.0 |
| | | 2 | 4.95 | 2.1 |
| | | 4 | 4.30 | NA |
| | | 6 | 1.07 | 0.33 |
| | | 8 | 2.95 | NA |
| SST | | | | |
| Fusion | | | | |
| Protein | | Sampling | | |
| Dose (mg/ | Dose | Time | Mean | Standard |
| kg) | Route | (hour) | (ng/mL) | Deviation |
| 27.1 | IV | 0.00 | 0.36 | NA |
| | | 0.05 | 756393.18 | 223856.37 |
| | | 0.13 | 743515.21 | 233145.74 |
| | | 0.25 | 634640.70 | 243150.70 |
| | | 0.50 | 649841.53 | 252801.38 |
| | | 0.75 | 560439.29 | 183395.09 |
| | | 1.25 | 480207.30 | 105178.15 |
| | | 2.00 | 493399.21 | 90422.74 |
| | | 3.00 | 416740.09 | 98435.97 |
| | | 4.00 | 366465.56 | 98751.94 |

TABLE 9
Pharmacokinetic Parameters of SST and SST Fusion Protein in Rat
after Intravenous Administration

| PK | | SST Fusion Protein | | SST | |
|---|---|---|---|---|---|
| Parameters | Unit | Mean | SD | Mean | SD |
| AUC₀₋ₜ | mg · h/mL | 1960799 | 427419 | 29.0 | 16.0 |
| AUC_{0-inf} | mg · h/mL | 4730184 | 1698725 | 43.8 | 20.1 |
| AUMC₀₋ₜ | mg · h²/mL | 3502133 | 775860 | 55.9 | 27.1 |
| AUMC_{0-inf} | mg · h²/mL | 36520105 | 19055736 | 173 | 49.9 |
| MRT_{IV} | h | 7.2 | 2.0 | 4.1 | 0.75 |
| CL | mL/kg · min | 0.11 | 0.049 | 510 | 234 |
| CL | mL/kg · h | 6.53 | 2.9 | NA | NA |
| Vdₛₛ | L/kg | 0.043 | 0.0058 | 2.19 | 1.3 |

We claim:

1. A fusion protein comprising:
an SST;
an L; and
an ALB,
wherein,
SST is a somatostatin, its analogue or derivative;
L is a spacer or a linker; and
ALB is an albumin, its analogue or variant.

2. The fusion protein of claim 1, selected from the group consisting of:
SST-(L)ₓ₁-ALB (I);
ALB-(L)ₓ₁-SST (II);
[SST-(L)ₓ₁]_{y1}-ALB (III);
ALB-[(L)ₓ₁-SST]_{y1} (IV);
[SST-(L)ₓ₁]_{y1}-ALB-[(L)ₓ₂-SST]_{y2} (V);
[SST-(L)ₓ₁]_{y1}-ALB-[(L)ₓ₂-SST]_{y2}-(L)ₓ₃-ALB (VI);
[SST-(L)ₓ₁]_{y1}-ALB-[(L)ₓ₂-SST]_{y2}-(L)ₓ₃-ALB-[(L)ₓ₄-SST]_{y3} (VII);
ALB-(L)ₓ₁-[SST-(L)ₓ₂]_{y1}-ALB (VIII);
ALB-(L)ₓ₁-[SST-(L)ₓ₂]_{y1}-ALB-[(L)ₓ₃-SST]_{y2}-(L)ₓ₁-ALB (IX); and
ALB-(L)ₓ₁-[SST-(L)ₓ₂]_{y1}-ALB-[(L)ₓ₃-SST]_{y2}-(L)ₓ₁-ALB-[(L)ₓ₄-SST]_{y3} (X);
wherein,
x1, x2, x3, x4, y1, y2, or y3 is independently zero or an integer selected from 1-10, provided that there is at least one L present in the nucleotide sequence encoding an albumin-somatostatin fusion protein.

3. The fusion protein of claim 1, wherein the SST is either naturally occurring or synthetically manufactured.

4. The fusion protein of claim 1 wherein the SST comprises one or more tandem repeats of a sequence encoding SST-14 or SST-28, represented by SEQ ID NOS: 17 or 18, respectively, or a sequence having at least 85% identity to either of these sequences.

5. The fusion protein of claim 1, wherein the SST is SST-14 or SST-28.

6. The fusion protein of claim 1, wherein L is either flexible or alpha helically structured polypeptide linker or spacer.

7. The fusion protein of claim 1, wherein L is a polypeptide having 2-100 amino acids.

8. The fusion protein of claim 6, wherein the polypeptide contains at least one GGGGS, A(EAAAK)₄A, (AP)ₙ, wherein n is an integer selected from 10-34, (G)₈, (G)₅, or any combination thereof.

9. The fusion protein of claim 1, wherein ALB is mammalian serum albumin.

10. The fusion protein of claim 1, wherein the mammalian serum albumin is SEQ ID NO: 25, or a sequence having at least 85% sequence identity thereto.

11. The fusion protein of claim 2, wherein x1, x2, x3, x4 are each independently an integer selected from 1-5.

12. The fusion protein of claim 2, wherein y1, y2, y3 are each independently an integer selected from 1-5.

13. A nucleotide sequence encoding a polypeptide comprising:
an SST;
an L; and
an ALB,
wherein,
SST is a somatostatin or its analogues or derivatives;
L is a spacer or a linker; and
ALB is an albumin or its analogues or variants.

14. The nucleotide sequence of claim 13, encoding a polypeptide that is selected from the group consisting of,
SST-(L)ₓ₁-ALB (I);
ALB-(L)ₓ₁-SST (II);
[SST-(L)ₓ₁]_{y1}-ALB (III);
ALB-[(L)ₓ₁-SST]_{y1} (IV);
[SST-(L)ₓ₁]_{y1}-ALB-[(L)ₓ₂-SST]_{y2} (V);
[SST-(L)ₓ₁]_{y1}-ALB-[(L)ₓ₂-SST]_{y2}-(L)ₓ₃-ALB (VI);
[SST-(L)ₓ₁]_{y1}-ALB-[(L)ₓ₂-SST]_{y2}-(L)ₓ₃-ALB-[(L)ₓ₄-SST]_{y3} (VII);
ALB-(L)ₓ₁-[SST-(L)ₓ₂]_{y1}-ALB (VIII);
ALB-(L)ₓ₁-[SST-(L)ₓ₂]_{y1}-ALB-[(L)ₓ₃-SST]_{y2}-(L)ₓ₁-ALB (IX); and
ALB-(L)ₓ₁-[SST-(L)ₓ₂]_{y1}-ALB-[(L)ₓ₃-SST]_{y2}-(L)ₓ₁-ALB-[(L)ₓ₄-SST]_{y3} (X);
wherein,
each of x1, x2, x3, x4, y1, y2, or y3 is independently zero or an integer selected from 1-10,
provided that there is at least one L present in the polypeptide.

15. The nucleotide sequence of claim 13, encoding the polypeptide sequence, wherein the SST comprises one or more tandem repeats of a sequence encoding SST-14 or SST-28, represented by SEQ ID NOS: 17 or 18, respectively, or a sequence having at least 85% identity to either SEQ ID NO: 17 or SEQ ID NO: 18.

16. A plasmid construct expressing an albumin-somatostatin fusion protein comprising the fusion protein of claim 1.

17. A bacterial host cell transformed with the plasmid construct of claim 16.

18. The fusion protein of claim 1 that is isolated and purified.

19. A method of treating a disease or disorder of endocrine release in a human subject by administering an effective amount of a pharmaceutical composition comprising the fusion protein of claim 1, wherein the disease or disorder of endocrine release is a condition that responds to the administration of somatostatin.

20. The method of claim 19, wherein the condition is a cancer selected from the group consisting of breast cancer, colorectal cancer, liver cancer, endocrine cancer, neuroendocrine cancers, pancreatic cancer, prostate cancer, brain cancer and lung cancer.

21. The method of claim 20, wherein the cancer expresses somatostatin receptor type 1, 2, 3, 4 or 5.
